(19) United States
(12) Patent Application Publication
(10) Pub. No.: US 2024/0158819 A1
(43) Pub. Date: May 16, 2024

(54) Use of Biological Enzyme for Preparing Orlistat Intermediate, and Preparation Method

(71) Applicant: ZEIN BIOTECHNOLOGY CO., LTD., CHONGQING (CN)

(72) Inventors: Tianshuai XU, CHONGQING (CN); Dayong GONG, CHONGQING (CN); Yumel XIAO, CHONGQING (CN); Zhanghong WANG, CHONGQING (CN); Zhichuan HUANG, CHONGQING (CN); Shan HUANG, CHONGQING (CN); Lei ZHANG, CHONGQING (CN); Xin GAO, CHONGQING (CN); Junwei SHEN, CHONGQING (CN)

(21) Appl. No.: 17/790,787

(22) PCT Filed: Jan. 6, 2021
(86) PCT No.: PCT/CN2021/070495

§ 371 Date: Jul. 5, 2022

(30) Foreign Application Priority Data
Jan. 7, 2020 (CN) 202010015510.1

(51) Int. Cl. C12P 7/42; C12N 9/04; C12N 15/62

(52) U.S. Cl. CPC C12P 7/42; C12N 9/0006; C12N 15/62; C07K 2319/00; C12Y 101/01002; C12Y 101/01047

(57) ABSTRACT

Provided is a method for biosynthesis of (R)-β-hydroxytetradecanoate compounds. The method comprises the following steps of: reacting β-carbonyl tetradecanoate in a ketoreductase and a glucose dehydrogenase or a fusion enzyme thereof, glucose, NADP⁺ and a buffer solution to obtain (R)-β-hydroxytetradecanoate compounds. The method is an enzyme-catalyzed biosynthesis method having simple operations, conventional equipment and an environmentally friendly process, and the prepared product has high purity, high yield and a high ee value.

### RELATED APPLICATIONS

This is a national phase application of PCT/CN2021/070495 filed Jan. 6, 2021, which claims the benefit of CN202010015510.1 filed Jan. 7, 2020, each of which is incorporated herein by reference in its entirety.

### SEQUENCE LISTING

The specification further incorporates by reference the Sequence Listing submitted herewith via EFS. The Sequence Listing text file, identified as Sequence Listing-EIC2021042001CN-US.txt, was created on Jan. 4, 2021 and then corrected on Aug. 18, 2023 to have the size of 25,773 bytes. The Sequence Listing, electronically filed herewith, does not extend beyond the scope of the specification filed Jul. 5, 2022 and thus does not give rise to new matter.

### FIELD OF THE INVENTION

The present invention belongs to the technical field of biopharmacy and biochemical engineering, and particularly relates to a method for biosynthesis of (R)-β-hydroxytetradecanoate compounds.

### BACKGROUND OF THE INVENTION

There are mainly two categories of drugs for treating obesity, that is antiobesity drugs acting on central nerves and antiobesity drugs without acting on the central nerves. The antiobesity drugs acting on the central nerves have obvious weight-reducing effect followed by large side effect and thus are largely limited in clinical application, wherein fenfluramine got off the market in 1997 due to the capability of causing pulmonary arterial hypertension and hypertrophic valve disorders; sibutramine was announced to stop production, sales and use by State Food and Drug Administration in 2010 due to the risk that serious cardiovascular diseases might be increased; and locaserin (trade name: Belviq) was approved for marketing by the FDA at June, 2012, still has the risk of increasing valvular diseases and adverse cardiovascular events and requires further verification in safety of clinical application.

Orlistat is a first antiobesity drug without acting on the central nerves and is an only one OTC antiobesity drug in the world at present. Since the orlistat came to the market in 1998, it has already become the first drug for treating the obesity due to obvious curative effect and high safety. Orlistat directly acts on gastrointestinal lipase, is significant in effect of inhibiting absorption of fats, is highly specific to a target, has no influence on other gastrointestinal enzymes, exerts the effect without systemic absorption, is very low in exposure amount of system drugs in a body, has the main side effect of gastrointestinal reactions and is excellent in safety. Orlistat has been sold for more than 20 years in the world, has been approved for marketing in more than 145 countries at present and has been verified by large clinical applications in excellent curative effect and safety.

The market demand on the orlistat is large, so that it is very important to find efficient synthetic methods for the orlistat and an intermediate thereof. (R)-β-hydroxytetradecanoate is an important intermediate for synthesizing the orlistat as a drug; and an optical purity of the intermediate is crucial. But how do we efficiently obtain optically pure (R)-β-hydroxytetradecanoate?

For example, Chinese Patent CN101538285B reported an asymmetric hydrogenation catalyzed by using a prepared (R)-metal catalyst [(R)-Ru (MeOBIPHEP)Cl2]2⋅NEt3; however, such chemical catalytic reaction requires to be conducted under the conditions with strong acid and high pressure of 60 bar only, and then a product with an ee value larger than 98.5% can be obtained. Such method has the deficiencies that: 1) an expensive (R)-ligand and noble metal catalyst are required; 2) the catalyst requires to be prepared while being used, and the process stability is difficultly guaranteed; 3) high-pressure hydrogenation equipment is required; 4) acid-resistant equipment is required; and 5) the ee value can be affected if the reaction is poorly controlled.

### OBJECTS AND SUMMARY OF THE INVENTION

Aiming to the above disadvantages in the prior art, the inventor uses a biological enzyme method for an improvement on system and then solves the problem of scale production of key intermediates of orlistat.

In view of this, the first objective of the present invention is to provide a novel use of a biological enzyme for preparing an orlistat intermediate. The biological enzyme can effectively act on β-carbonyl tetradecanoate as a substrate, so as to prepare the orlistat intermediate with a high purity.

In order to achieve the above objective, the present invention provides the following technical solution:
a use of a biological enzyme for preparing an orlistat intermediate (final product) is provided. A substrate of the biological enzyme action is β-carbonyl tetradecanoate (raw material) with a structural formula shown as I; the orlistat intermediate is (R)-β-hydroxytetradecanoate with a structural formula shown as II; in the structural formula I, R refers to a saturated alkyl group having 1-3 carbon atoms; and in the structural formula II, R refers to a saturated alkyl group having 1-3 carbon atoms.

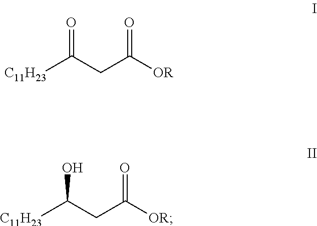

The biological enzyme is a ketoreductase having an amino acid sequence shown as SEQ ID NO: 1 and an amino acid sequence of 90% or more homology thereto, is from Singulisphaera acidiphila, is coded as WP_015245403.1 in an NCBI data bank and belongs to a short chain dehydrogenase family; and/or the biological enzyme has an amino acid sequence shown as SEQ ID NO: 2 and an amino acid sequence of 90% or more homology thereto, is from Sphingomonas echinoides, is coded as WP_010403640.1 in the NCBI data bank and belongs to a short chain dehydrogenase family; and/or the biological enzyme has an amino acid sequence shown as SEQ ID NO: 4 and an amino acid sequence of 80% or more identity thereto, is from Rhodotorula toruloides, is a kind of truncated proteins coded as EGU12837.1 protein in the NCBI data bank and belongs to a short chain dehydrogenase family. Or the biological enzyme is a fusion enzyme of the ketoreductase and a glucose dehydrogenase; and an amino acid sequence of the fusion enzyme is shown as SEQ ID NO: 8 and/or SEQ ID NO: 9. The inventor is surprised to find that a natural biological enzyme or a biological enzyme modified by gene/protein engineering has very good catalytic effect to the substrate, and a final product with high optical purity is obtained.

Further, the biological enzyme having the amino acid sequence of 90% or more identity/homology to SEQ ID NO: 1 and/or SEQ ID NO: 2, of 80% or more identity/homology to SEQ ID NO: 4 and having the amino acid sequence shown as SEQ ID NO: 8 and/or SEQ ID NO: 9 may further be applied to preparing the orlistat intermediate.

Further, in the fusion enzyme of the ketoreductase and the glucose dehydrogenase, the ketoreductase is linked to the glucose dehydrogenase through a linker.

Further, an amino acid sequence of the linker is shown as SEQ ID NO: 5.

Further, the fusion enzyme of the ketoreductase and the glucose dehydrogenase is ketoreductase-linker-glucose dehydrogenase or glucose dehydrogenase-linker-ketoreductase; and optionally, an amino acid sequence of the glucose dehydrogenase is shown as SEQ ID NO: 3.

Further, any of the forgoing biological enzymes is enzyme powder, an enzyme solution and/or an immobilized enzyme.

Further, in the structural formula I, R is any one of methyl, ethyl, n-propyl or isopropyl; and in the structural formula II, R is any one of the methyl, the ethyl, the n-propyl or the isopropyl.

The second objective of the present invention is to provide a fusion enzyme. The fusion enzyme is a fusion enzyme of the ketoreductase and the glucose dehydrogenase; and an amino acid sequence of the fusion enzyme is shown as SEQ ID NO: 8 and/or SEQ ID NO: 9.

Further, in the fusion enzyme, the ketoreductase is linked to the glucose dehydrogenase through a linker; and optionally, an amino acid sequence of the linker is shown as SEQ ID NO: 5.

Further, the fusion enzyme is the ketoreductase-linker-glucose dehydrogenase or the glucose dehydrogenase-linker-ketoreductase.

Optionally, an amino acid sequence of the ketoreductase is shown as SEQ ID NO: 1 and/or SEQ ID NO: 2 and/or SEQ ID NO: 4 and an amino acid sequence of 80% or more identity/homology thereto.

Optionally, an amino acid sequence of the glucose dehydrogenase is shown as SEQ ID NO: 3.

The third objective of the present invention is to provide a nucleotide sequence for coding the fusion enzyme and a method for constructing the nucleotide sequence.

A nucleotide sequence of the fusion enzyme is shown as SEQ ID NO: 6 or SEQ ID NO: 7.

Further, the method for constructing the nucleotide sequence comprises the steps of: inserting a linker sequence shown as SEQ ID NO: 5 into 3′ end of a gene fragment of the glucose dehydrogenase shown as SEQ ID NO: 3, and linking a gene fragment of the ketoreductase shown as SEQ ID NO: 4 behind the linker sequence to form a recombinant plasmid having a nucleotide sequence shown as SEQ ID NO: 6.

Further, the nucleotide sequence (SEQ ID NO: 6) of the fusion enzyme is linked to a vector pET28a at digestion sites NdeI and XhoI at two ends to form a double-enzyme fusion expression plasmid pET28a-G3790, and the double-enzyme fusion expression plasmid pET28a-G3790 is transformed to Escherichia coli for screening, inoculation and culture to obtain thalli; and the thalli are crushed and centrifuged to obtain a crude enzyme solution, and the crude enzyme solution is freeze-dried to obtain enzyme powder.

Further, the method for constructing the nucleotide sequence comprises the steps of: inserting a linker sequence shown as SEQ ID NO: 5 into 3′ end of a gene fragment of the ketoreductase shown as SEQ ID NO: 4, and linking a gene fragment of the glucose dehydrogenase shown as SEQ ID NO: 3 behind the linker sequence to form a nucleotide sequence shown as SEQ ID NO: 7.

Further, the nucleotide sequence (SEQ ID NO: 7) of the fusion enzyme is linked to a vector pET28a at digestion sites NdeI and XhoI at two ends to form a double-enzyme fusion expression plasmid pET28a-G3790, and the double-enzyme fusion expression plasmid pET28a-G3790 is transformed to Escherichia coli for screening, inoculation and culture to obtain thalli; and the thalli is crushed and centrifuged to obtain a crude enzyme solution, and the crude enzyme solution is freeze-dried to obtain enzyme powder.

The fourth objective of the present invention is to provide a composition. In the composition, two can form a synergic relationship of the enzyme and the substrate, and a product with a structural formula shown as II is obtained.

In order to achieve the above objective, the present invention provides the following technical solution:
provided is the composition containing any one of the biological enzymes and the substrate, wherein the substrate is β-carbonyl tetradecanoate. the β-carbonyl tetradecanoate has the structural formula shown as I; the biological enzyme is the ketoreductase or the fusion enzyme of the ketoreductase and the glucose dehydrogenase; the amino acid sequence of the ketoreductase is shown as SEQ ID NO: 1 and/or SEQ ID NO: 2 and/or SEQ ID NO: 4 and an amino acid sequence of 80% or more identity/homology thereto; the amino acid sequence of the fusion enzyme is shown as SEQ ID NO: 8 and/or SEQ ID NO: 9;

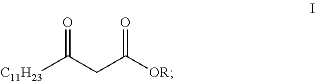

and
in the structural formula I, R refers to a saturated alkyl group having 1-3 carbon atoms.

Further, in the fusion enzyme, the ketoreductase is linked to the glucose dehydrogenase through a linker; and optionally, an amino acid sequence of the linker is shown as SEQ ID NO: 5.

Further, the fusion enzyme is the ketoreductase-linker-glucose dehydrogenase or the glucose dehydrogenase-linker-ketoreductase.

Further, the amino acid sequence of the glucose dehydrogenase is shown as SEQ ID NO: 3.

Further, the biological enzyme is enzyme powder, an enzyme solution and/or an immobilized enzyme.

Further, in the structural formula I, R is any one of methyl, ethyl, n-propyl or isopropyl; and in the structural formula II, R is any one of the methyl, the ethyl, the n-propyl or the isopropyl.

Further, in the composition, wherein a weight ratio of the biological enzyme to the substrate is 1:(1.1-150).

Preferably, in the composition, the weight ratio of the biological enzyme to the substrate is one of 1:20, 1:30, 1:40, 1:50, 1:60, 1:70, 1:80, 1:90, 1:100, 1:110, 1:120, 1:130, 1:140 and 1:150.

The fifth objective of the present invention is to provide a reaction system for preparing (R)-β-hydroxytetradecanoate. By using the reaction system, the (R)-β-hydroxytetradecanoate may be obtained without stringent conditions of a high temperature and a high pressure.

In order to achieve the above objective, the present invention provides the following technical solution:
the reaction system comprises a substrate shown as I, a biological enzyme, glucose, a glucose dehydrogenase, NADP+ and a buffer solution; and here, the biological enzyme is a ketoreductase having an amino acid sequence shown as SEQ ID NO: 1 and/or SEQ ID NO: 2 and/or SEQ ID NO: 4 and an amino acid sequence of 80% or more identity/homology thereto. Or the reaction system comprises the substrate shown as I, the biological enzyme, the glucose, NADP+ and the buffer solution; here, the biological enzyme is a fusion enzyme of the ketoreductase and the glucose dehydrogenase; an amino acid sequence of the ketoreductase is shown as SEQ ID NO: 1 and/or SEQ ID NO: 2 and/or SEQ ID NO: 4 and an amino acid sequence of 80% or more identity/homology thereto; and an amino acid sequence of the fusion enzyme of the ketoreductase and the glucose dehydrogenase is shown as SEQ ID NO: 8 and/or SEQ ID NO: 9.

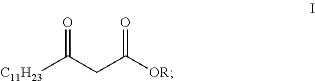

and
in the structural formula I, R refers to a saturated alkyl group having 1-3 carbon atoms.

Further, the amino acid sequence of the glucose dehydrogenase is shown as SEQ ID NO: 3. Further, in the fusion enzyme, the ketoreductase is linked to the glucose dehydrogenase through a linker; and optionally, an amino acid sequence of the linker is shown as SEQ ID NO: 5.

Further, the fusion enzyme is the ketoreductase-linker-glucose dehydrogenase or the glucose dehydrogenase-linker-ketoreductase.

Further, the amino acid sequence of the glucose dehydrogenase is shown as SEQ ID NO: 3.

Further, the biological enzyme is enzyme powder, an enzyme solution and/or an immobilized enzyme.

Further, in the structural formula I, R is any one of methyl, ethyl, n-propyl or isopropyl; and in the structural formula II, R is any one of the methyl, the ethyl, the n-propyl or the isopropyl.

Further, a pH value of the reaction system is 6.0-8.0.

Further, in the reaction system, the buffer solution is a PBS buffer solution or a Tris-HCl buffer solution.

Further, in the reaction system, a concentration of the substrate is 20-150 g/L.

Further, the reaction system is characterized in that a concentration of NADP+ is 0.1-0.5 g/L.

Further, in the reaction system, a molar ratio of a compound I to the glucose is 1:(1.2-4).

Further, in the reaction system, a concentration of the buffer solution is 0.01-0.5 mol/L.

Further, in the reaction system, the reaction time does not exceed 15 h, and a reaction liquid is obtained.

The sixth objective of the present invention is to provide a method of preparing the orlistat intermediate. The method is suitable for industrial scale production.

A reaction formula of a method for biosynthesis of (R)-(β-hydroxytetradecanoate compounds is as follows:

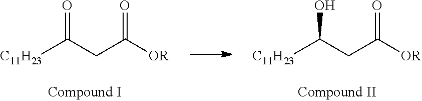

In the compound I and the compound II, R refers to a saturated alkyl group having 1-3 carbon atoms; and preferably, R is any one of the methyl, the ethyl, the n-propyl or the isopropyl.

The biosynthesis method comprises the steps of: reacting the compound I in the ketoreductase, the glucose, the glucose dehydrogenase and NADP⁺ or in the fusion enzyme of the ketoreductase and the glucose dehydrogenase, the glucose and NADP⁺ to obtain the compound II.

In an implementation solution of the present invention, the biosynthesis method comprises the following steps of: putting the compound I in the buffer solution, and then adding the ketoreductase, or the fusion enzyme of the ketoreductase and the glucose dehydrogenase, the glucose, the glucose dehydrogenase (the glucose dehydrogenase does not require to be added while the fusion enzyme of the ketoreductase and the glucose dehydrogenase is added) and NADP⁺ to a mixture to obtain a mixed solution; stirring the mixed solution at 20-40° C. for reaction, adjusting a pH value by a NaOH aqueous solution in the whole process, and monitoring a conversion rate of reaction by using liquid chromatography, and ending the reaction until the conversion rate reaches 99% or above; adding an extracting solvent for extraction; combining organic phases, conducting concentration under reduced pressure, cooling and crystallization, and precipitating a product to obtain a white solid which is the compound II; and optionally, further conducting recrystallization with normal hexane to obtain a product with a higher purity.

In some implementation solutions of the present invention, a concentration of the NaOH aqueous solution may be 2 M.

In some implementation solutions of the present invention, a pH value of the buffer solution is 6.0-8.0; preferably, the buffer solution is a PBS (i.e., phosphate) buffer solution or a Tris-HCl buffer solution; more preferably, the buffer solution is 0.01-0.5 mol/L PBS phosphate buffer solution or 0.01-0.5 mol/L Tris-HCl buffer solution.

In an implementation solution of the present invention, in the reaction process, a pH value range is controlled at 7.0-7.5.

In an implementation solution of the present invention, the amino acid sequence of the ketoreductase is shown as SEQ ID NO: 1, SEQ ID NO: 2 and SEQ ID NO: 4 of this application; the amino acid sequence of the fusion enzyme of the ketoreductase and the glucose dehydrogenase is shown as SEQ ID NO: 8 and/or SEQ ID NO: 9; the ketoreductase or the fusion enzyme of the ketoreductase and the glucose dehydrogenase may be in a form of the enzyme powder, the enzyme solution or the immobilized enzyme; and the amino acid sequence of the glucose dehydrogenase is shown as SEQ ID NO: 3, and the glucose dehydrogenase may be in a form of the enzyme powder, the enzyme solution or the immobilized enzyme.

In an implementation solution of the present invention, NADP⁺ refers to nicotinamide adenine dinucleotide phosphate which is an oxidized form of reduced coenzyme II (NADPH); and in the mixed solution, the concentration of NADP⁺ is 0.1-0.5 g/L.

In an implementation solution of the present invention, the concentration of the compound I in the mixed solution is 20-150 g/L.

In an implementation solution of the present invention, a molar ratio of the compound I to the glucose in the mixed solution is 1:(1.2-4).

In some implementation solutions of the present invention, preferably, a temperature of the reaction is kept at 35° C.

In some implementation solutions of the present invention, the extracting solution is used for extraction twice and is anhydrous ethanol or ethyl acetate.

The technical solution of the above contents are summarized, specifically:

the method for preparing the orlistat intermediate based on the reaction system comprises the steps of: stirring the reaction system at 20-40° C. for reaction to obtain a reaction liquid of the orlistat intermediate, wherein an intermediate of the orlistat intermediate is the (R)-β-hydroxytetradecanoate with the structural formula shown as II;

Further, in the method, the NaOH aqueous solution is used as a pH regulator.

Further, in the method, the reaction time does not exceed 15 h.

Further, in the method, a solvent is used in the reaction liquid to extract the orlistat intermediate.

Further, in the method, the solvent is the anhydrous ethanol or the ethyl acetate.

Further, in the method, an obtained extract is concentrated under reduced pressure, cooled and crystallized, and a white crystalline solid, which is the compound II, is obtained.

Provided is orlistat prepared with the orlistat intermediate.

The Beneficial Effects Resulting from the Present Invention:

The biological enzyme used in the present invention may tolerate the concentration of the substrate reaching up to 150 g/L, and the activity of the enzyme is not limited by the substrate or the product.

A method for preparing the orlistat intermediate of the present invention is a biological enzyme method having mild conditions, conventional equipment and simple operations; and by using the method, the quantity of produced three wastes is small, no heavy metal pollution is caused, the environment is protected, and industrial production is facilitated.

By using the enzyme catalyzing process of the present invention, the conversion rate reaches up to 99% or above, and a chiral ee value may reach up 99% or above; the reaction may be fully completed within 15 h, and the concentration of the product is high; and nearly full conversion of the substrate may simplify the aftertreatment step of the reaction liquid, and the high-purity product may be obtained only requiring simple extraction and crystallization steps, so that the production cost is greatly lowered.

### BRIEF DESCRIPTION OF FIGURES

FIG. 1 is an HPLC chromatogram of a measured conversion rate according to Embodiment 5;

FIG. 2 is an HPLC chromatogram of a purity of a product according to Embodiment 5;

FIG. 3 is an HPLC chromatogram of a chiral purity of a product according to Embodiment 5;

FIG. 4 is a plasmid map of an expression plasmid for a fusion enzyme G3790;

FIG. 5 is a plasmid map of an expression plasmid for a fusion enzyme 3790G;

FIG. 6 is a diagram showing protein bands of a fusion enzyme G3790; and

FIG. 7 is a diagram showing protein bands of a fusion enzyme 3790G.

### DETAILED DESCRIPTION OF THE EMBODIMENTS

Listed embodiments are for better describing the present invention, but are not regarded as limitations for the contents of the present invention. So, nonessential improvements and adjustments made on the embodiments by those familiar with the art according to the above contents of the present invention should still fall within the protection scope of the present invention.

The present invention relates to that β-carbonyl tetradecanoate is reduced to (R)-β-hydroxytetradecanoate under the catalysis of the biological enzyme. A reaction formula is as follows:

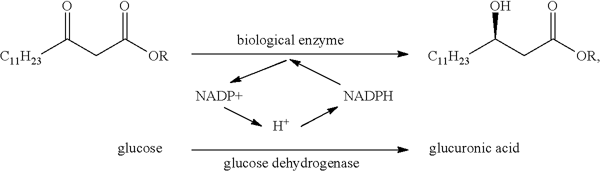

β-carbonyl tetradecanoate, used as the substrate, is catalyzed by a certain enzyme in an environment containing a NADP⁺ coenzyme, glucose and a glucose dehydrogenase to obtain (R)-β-hydroxytetradecanoate with a very high chiral purity, wherein a catalyzing enzyme generally belongs to a short chain dehydrogenase family and may further be a fusion enzyme of different enzymes, for example, a fusion enzyme of reductase and the glucose dehydrogenase in some embodiments of the present invention; and wherein the glucose is dehydrogenized under the effect of the glucose dehydrogenase to obtain H⁺, and then the NADP⁺ coenzyme carries H⁺ to participate to reduction reaction of the β-carbonyl tetradecanoate to obtain β-hydroxytetradecanoate.

A ketoreductase JR3789 of the present invention is from Singulisphaera acidiphila, is coded as WP_015245403.1 in an NCBI data bank, belongs to the short chain dehydrogenase family and has an amino acid sequence shown as SEQ ID NO: 1 in a size of 249 amino acids.

The amino acid sequence SEQ ID NO: 1 is shown as follows:

“MGKLDNKVAVITGGNSGMGLATAQRFVSEGAYVFITGRRQAELDKAVDL
IGKNVTGVQGDVSNLADLDRLYATVKEQKGRVDVLFANAGVGELAPLGSI
TEEQFDKVFNINVRGLLFTVQKALPLFQDGGSIILNASIASIKGMPAFSV
YSASKAAVRSFARSWTVDLKGRKIRINTLSPGPIDTPILSGLASTEEELK
QVKADLAAQVPLGRMGTSDEIANVALFLASDDSSYVTGIELFVDGGMAQ
I”.

A ketoreductase JR37150 of the present invention is from Sphingomonas echinoides, is coded as WP_010403640.1 in the NCBI data bank, belongs to the short chain dehydrogenase family and has an amino acid sequence shown as SEQ ID NO: 2 in a size of 259 amino acids.

The amino acid sequence SEQ ID NO: 2 is shown as follows:

“MARLAGKVALVTGGASVPGLGSATAIRFAQEGATVYLTDRDLAGAQAVA
AQITAAGGRATALEHDVTSEADWDRVLAAIDAAEGRLDILVNNAGIAVLG
PLEDVTAADFLRQNDVNLNSVFHGSKRALVMMRRPGDGGTARGGSIINIS
SVAGLIGVPGCGSYAASKGGVRLFSKVVALEGAADGVRCNSVHPGMIATN
IQGVALEDNAANFDAVMALIPMVRMGEPEDIANMNLFLASDESRYITGAE
FVVDGGMTAR”.

The glucose dehydrogenase (GDH) of the present invention is from Bacillus subtilis QB928, is coded as AFQ56330.1 in the NCBI data bank, belongs to the short chain dehydrogenase family and has an amino acid sequence shown as SEQ ID NO: 3 in a size of 263 amino acids.

The amino acid sequence SEQ ID NO: 3 is shown as follows:

“MYMYPDLKGKVVAITGAASGLGKAMAIRFGKEQAKVVINYYSNKQDPNE
VKEEVIKAGGEAVVVQGDVTKEEDVKNIVQTAIKEFGTLDIMINNAGLEN
PVPSHEMPLKDWDKVIGTNLTGAFLGSREAIKYFVENDIKGNVINMSSVH
EVIPWPLFVHYAASKGGIKLMTETLALEYAPKGIRVNNIGPGAINTPINA
EKFADPKQKADVESMIPMGYIGEPEEIAAVAAWLASKEASYVTGITLFAD
GGMTQYPSFQAGRG”.

A ketoreductase JR3790 of the present invention is from Rhodotorula toruloides, is a kind of truncated proteins (of 70-371 bits) coded as EGU12837.1 protein in the NCBI data bank, belongs to the short chain dehydrogenase family and has an amino acid sequence shown as SEQ ID NO: 4 in a size of 248 amino acids.

The amino acid sequence SEQ ID NO: 4 is shown as follows:

“MSSPAPTVYVISGASRGIGFAITSILAQHDNVLIFAGARDLKSAQLNEL
AQKSSGKVIPVKLESTSVEDAAALAKVVEEKAGKVDYVLAVAGISQSTDP
IAQVSLDDVRRHFEVNTIGPLVLFQALLPLTTKSTAPHFIVVSTIAGSIA
SMPQVTFPVSAYAISKTAVNSAVGRIAIEHPDLDAFVCHPGFVSSDMVKQ
FAEKTGAPLSDFESFGMITPEESAASLVKLFDEAKKETHSGKFFNVDG
T”.

A sequence of the linker is shown as SEQ ID NO: 5:

“EFEEEEKKKQQEEEAERLRRIQEEMEKERKRREEDEERRRKEEEERRMK
LEMEAKRKQEEEERKKREDDEKRKKKKL”.

HPLC detection conditions: OD-H chromatographic column; a mobile phase is normal hexane: isopropanol=98:2; a flow rate is 1.0 mL/min. A detection instrument has a specification as follows: DAD1C, Sig=210,4 Ref=360,100.

The biological enzymes (SEQ ID NO: 1, SEQ ID NO: 2) and the glucose dehydrogenase (SEQ ID NO: 3) are all synthesized by Nanjing GenScript Biotech Corporation and are commercialized.

### Example 1

1 g of β-carbonyl methyl tetradecanoate and 1.5 g of glucose were weighed and put in a 100 mL three-necked flask, and then 50 mL of a PBS buffer solution with a concentration of 50 mM and a pH value of 7.0 were added; the three-necked flask was put into a thermostat water bath, a stirring speed was adjusted to 800 rpm, and a temperature was adjusted to 35° C.; then 10 mg of NADP⁺, 25 mg of glucose dehydrogenase (GDH) powder (shown as SEQ ID NO: 3) and 100 mg of ketoreductase JR3789 powder (shown as SEQ ID NO: 1) were added to obtain a mixed reaction liquid; a pH value was maintained at 7.0-7.5 by using a NaOH solution with a concentration of 2 M; the temperature was maintained at 35° C.; the reaction process was monitored by HPLC; the reaction ended after 9 h; and a conversion rate was measured to be larger than 99%.

After reaction was completed, the temperature was raised to 60° C. firstly, kept for 15 min and then cooled to 20-25° C.; 80 mL of ethyl acetate was added for extraction and stirring for 20 min; filtering was conducted; and a filtrate was layered to take an organic phase. An aqueous phase was extracted once by using 50 mL of ethyl acetate again, and layering was conducted to take an organic phase. The organic phases were combined; a product was concentrated under reduced pressure and then was slowly cooled; and a product was precipitated to obtain a crude product with a purity of 98.10% and an optical purity of 98.45%. 2 times normal hexane by volume was added to the crude product for dissolution with heating; a product was cooled and crystallized; crystals were collected and dried at a room temperature; 0.89 g of white crystal product was collected and measured to have a purity of 99.99%, an ee value of 99.91% and a total yield of 89%.

### Example 2

1 g of β-carbonyl ethyl tetradecanoate and 1.5 g of glucose were weighed and put in a 100 mL three-necked flask, and then 50 mL of a PBS buffer solution with a concentration of 50 mM and a pH value of 7.0 were added; the three-necked flask was put into a thermostat water bath, a stirring speed was adjusted to 900 rpm, and a temperature was adjusted to 35° C.; then 10 mg of NADP+, 35 mg of glucose dehydrogenase (GDH) powder (shown as SEQ ID NO: 3) and 150 mg of ketoreductase JR3789 powder (shown as SEQ ID NO: 1) were added to obtain a mixed reaction liquid; a pH value was maintained at 7.0-7.5 by using a NaOH solution with a concentration of 2 M; the temperature was maintained at 35° C.; the reaction process was monitored by HPLC; the reaction ended after 10 h; and a conversion rate was measured to be larger than 99%.

After reaction was completed, the temperature was raised to 60° C. firstly, kept for 15 min and then cooled to 20-25° C.; 80 mL of ethyl acetate was added for extraction and stirring for 20 min; filtering was conducted; and a filtrate was layered to take an organic phase. An aqueous phase was extracted once by using 50 mL of ethyl acetate again, and layering was conducted to take an organic phase. The organic phases were combined; a product was concentrated under reduced pressure and then was slowly cooled; and a product was precipitated to obtain 0.84 g of white product with a purity of 98.42%, an ee value of 98.15% and a total yield of 84%.

### Example 3

5 g of β-carbonyl methyl tetradecanoate and 7.5 g of glucose were weighed and put in a 100 mL three-necked flask, and then 50 mL of a PBS buffer solution with a concentration of 50 mM and a pH value of 7.0 were added; the three-necked flask was put into a thermostat water bath, a stirring speed was adjusted to 800 rpm, and a temperature was adjusted to 35° C.; then 50 mg of NADP+, 125 mg of glucose dehydrogenase (GDH) powder (shown as SEQ ID NO: 3) and 500 mg of ketoreductase JR3789 powder (shown as SEQ ID NO: 1) were added to obtain a mixed reaction liquid; a pH value was maintained at 7.0-7.5 by using a NaOH solution with a concentration of 2 M; the temperature was maintained at 35° C.; the reaction process was monitored by HPLC; the reaction ended after 13 h; and a conversion rate was measured to be larger than 99%.

After reaction was completed, the temperature was raised to 60° C. firstly, kept for 15 min and then cooled to 20-25° C.; 80 mL of ethyl acetate was added for extraction and stirring for 20 min; filtering was conducted; and a filtrate was layered to take an organic phase. An aqueous phase was extracted once by using 50 mL of ethyl acetate again, and layering was conducted to take an organic phase. The organic phases were combined; a product was concentrated under reduced pressure and then was slowly cooled; and a product was precipitated to obtain 4.65 g of white product with a purity of 98.12%, an ee value of 98.45% and a total yield of 93%.

### Example 4

5 g of β-carbonyl methyl tetradecanoate and 7.5 g of glucose were weighed and put in a 100 mL three-necked flask, and then 50 mL of a PBS buffer solution with a concentration of 50 mM and a pH value of 7.0 were added; the three-necked flask was put into a thermostat water bath, a stirring speed was adjusted to 800 rpm, and a temperature was adjusted to 35° C.; then 50 mg of NADP+, 300 mg of glucose dehydrogenase (GDH) powder (shown as SEQ ID NO: 3) and 800 mg of ketoreductase JR37150 powder (shown as SEQ ID NO: 2) were added to obtain a mixed reaction liquid; a pH value was maintained at 7.0-7.5 by using a NaOH solution with a concentration of 2 M; the temperature was maintained at 35° C.; the reaction process was monitored by HPLC; the reaction ended after 35 h; and a conversion rate was measured to be larger than 99%.

After reaction was completed, the temperature was raised to 60° C. firstly, kept for 15 min and then cooled to 20-25° C.; 100 mL of ethyl acetate was added for extraction and stirring for 20 min; filtering was conducted; and a filtrate was layered to take an organic phase. An aqueous phase was extracted once by using 60 mL of ethyl acetate again, and layering was conducted to take an organic phase. The organic phases were combined; a product was concentrated under reduced pressure and then was slowly cooled; and a product was precipitated to obtain 4.55 g of white product with a purity of 98.62%, an optical purity of 99.66% and a total yield of 91%.

### Example 5

7.5 g of β-carbonyl methyl tetradecanoate and 11.25 g of glucose were weighed and put in a 100 mL three-necked flask, and then 50 mL of a PBS buffer solution with a concentration of 50 mM and a pH value of 7.0 were added; the three-necked flask was put into a thermostat water bath, a stirring speed was adjusted to 800 rpm, and a temperature was adjusted to 35° C.; then 150 mg of NADP+, 200 mg of glucose dehydrogenase (GDH) powder (shown as SEQ ID NO: 3) and 750 mg of ketoreductase JR3789 powder (shown as SEQ ID NO: 1) were added to obtain a mixed reaction liquid; a pH value was maintained at 7.0-7.5 by using a NaOH solution with a concentration of 2 M; the temperature was maintained at 35° C.; the reaction process was monitored by HPLC; the reaction ended after 15 h; and a conversion rate was measured to be larger than 99%. Detection results are shown in Table 1, and an HPLC chromatogram was shown as FIG. 1.

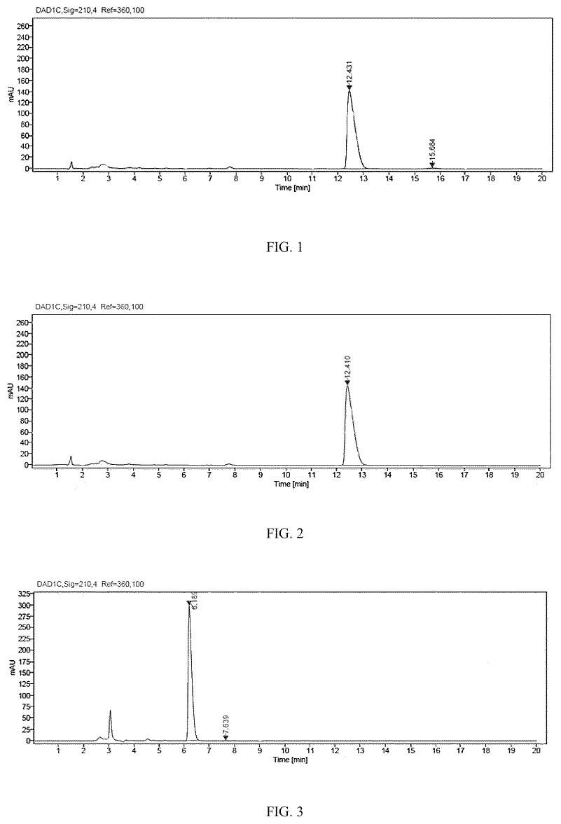

After reaction was completed, the temperature was raised to 60° C. firstly, kept for 15 min and then cooled to 20-25° C.; 100 mL of ethyl acetate was added for extraction and stirring for 20 min; filtering was conducted; and a filtrate was layered to take an organic phase. An aqueous phase was extracted once by using 80 mL of ethyl acetate again, and layering was conducted to take an organic phase. The organic phases were combined; a product was concentrated under reduced pressure and then was slowly cooled; and a product was precipitated to obtain a crude product with a purity of 99.60% and an ee value of 98.68%. 2 times normal hexane by volume was added to the crude product for dissolution with heating; a product was crystallized with cooling; crystals were filtered and dried at a room temperature; 6.53 g of white crystal product was collected and measured to have a purity of 99.99%, an ee value of 99.86% and a total yield of 87%. Detected data of purities of the product is shown in Table 2, and an HPLC chromatogram was shown as FIG. 2; and data of chiral purities of the product is shown in Table 3, and an HPLC chromatogram was shown as FIG. 3.

TABLE 1
Data table for measured conversion rates

| RT (min) | Type | Width (min) | Area | Height | Area |
|---|---|---|---|---|---|
| 12.431 | BB | 1.86 | 3001.36 | 142.52 | 99.06 |
| 15.684 | BBA | 0.79 | 28.40 | 1.48 | 0.94 |
| | | Sum | 3029.76 | | |

TABLE 2
Data table for measured purities of product

| RT (min) | Type | Width (min) | Area | Height | Area |
|---|---|---|---|---|---|
| 12.410 | BB | 1.36 | 3062.49 | 145.21 | 100.00 |
| | | Sum | 3062.49 | | |

TABLE 3
Data table for measured chiral purities of product

| RT (min) | Type | Width (min) | Area | Height | Area |
|---|---|---|---|---|---|
| 6.189 | BBA | 0.52 | 2944.36 | 298.07 | 99.93 |
| 7.639 | BB | 0.30 | 2.00 | 0.23 | 0.07 |
| | | Sum | 2946.336 | | |

### Example 6

### (1) Construction of Expression Plasmid for Fusion Enzyme G3790

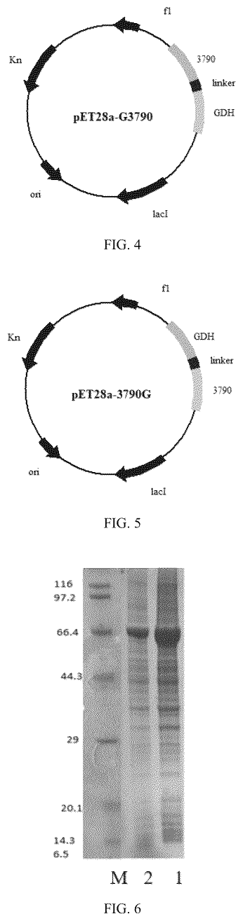

A gene sequence of a linker (SEQ ID NO: 5) was inserted into 3′ end of a gene fragment of glucose dehydrogenase (SEQ ID NO: 3), then a gene fragment of a ketoreductase JR3790 (SEQ ID NO: 4) was linked behind the gene sequence of the linker to form a sequence (SEQ ID NO: 6) of a fusion enzyme G3790 (synthesized by Nanjing GenScript Biotech Corporation), and the sequence was linked to a vector pET28a at digestion sites NdeI and XhoI at two ends to form a double-enzyme fusion expression plasmid pET28a-G3790, wherein a plasmid map of a recombinant plasmid is shown as FIG. 4.

### (2) Construction of Expression Plasmid for Fusion Enzyme 3790G

A gene sequence of a linker (SEQ ID NO: 5) was inserted into 3′ end of a gene fragment of a ketoreductase JR3790 (SEQ ID NO: 4), then a gene fragment of the glucose dehydrogenase (GDH) (SEQ ID NO: 3) was linked behind the gene sequence of the linker to form a sequence (SEQ ID NO: 7) of a fusion enzyme (synthesized by Nanjing GenScript Biotech Corporation), and the sequence was linked to a vector pET28a at the digestion sites NdeI and XhoI at two ends to form a double-enzyme fusion expression plasmid pET28a-3790G, wherein a plasmid map of a recombinant plasmid is shown as FIG. 5.

### (3) Preparation of Fusion Enzyme G3790 and Fusion Enzyme 3790G

The constructed fusion enzyme expression plasmids pET28a-G3790 and pET28a-3790G were transformed to competent E. coli BL21(DE3) strains of Escherichia coli; the strains were screened to obtain positive cloned transformants; monoclones containing the recombinant plasmids were selected to be inoculated to test tubes containing 5 mL of LB culture medium (100 μg/ml kanamycin) for culture at 37° C. and 200 rpm overnight; bacterial solutions with an inoculability quantity of 2% were transferred into triangular shake flasks containing 1 L of LB culture medium for culture at 37° C. and 200 rpm until OD600 reached about 0.6; isopropanol-β-D-thiogalactoside (IPTG) inducers (with a final concentration of 0.3 mM) were added for continuous culture for 12 h at 25° C.; and products were centrifuged, and thalli were collected. The thalli were resuspended after being diluted with PBS (pH=7.0), then subjected to ultrasonication and centrifuged to obtain supernates which was crude enzyme solutions.

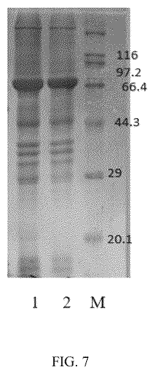

The crude enzyme solutions obtained by expression were subjected to protein band identification with SDS-polyacrylamide gel electrophoresis (SDS-PAGE). FIG. 6 shows a protein electrophoretogram for a G3790 fusion enzyme, wherein a lane M is protein Marker (GenScript); a lane 1 is total proteins after whole cell disruption; and a lane 2 is a supernate obtained after whole cell disruption and centrifugation; and wherein the fusion enzyme should have a theoretical molecular weight of 67 kDa and an amino acid sequence shown as SEQ ID NO: 8. FIG. 7 shows a protein electrophoretogram for a 3790G fusion enzyme, wherein a lane M is protein Marker (GenScript); a lane 1 is total proteins after whole cell disruption; and a lane 2 is a supernate obtained after whole cell disruption and centrifugation; and wherein both the fusion enzymes should have a theoretical molecular weight of 67 kDa and an amino acid sequence shown as SEQ ID NO: 9. The two fusion enzymes are both soluble proteins and have molecular weights approaching the corresponding theoretical molecular weights.

The crude enzyme solutions were prefreezed overnight and then freeze-dried for 24-36 h to obtain powder of the fusion enzyme G3790 and powder of the fusion enzyme 3790G.

### (4) Preparation of Orlistat Intermediate

7.5 kg of β-carbonyl methyl tetradecanoate and 9.375 kg of glucose were weighed and put in a 100 mL glass reactor, and then 25 L of a PBS buffer solution with a concentration of 50 mM and a pH value of 7.0 were added; a temperature of the reactor was controlled to keep at 35° C. by a high-low temperature cycling all-in-one machine; a mechanical stirring speed was adjusted to 180 rpm; then 18 g of NADP+ and 800 g of 3790G enzyme powder were added to obtain a mixed reaction liquid; a pH value was maintained at 7.0-7.5 by using a NaOH solution with a concentration of 2 M; the reaction process was monitored by HPLC; the reaction ended after 13 h; and a conversion rate was measured to be larger than 99%.

After the reaction was ended, the temperature was raised to 60° C. firstly, kept for 30 min and then cooled to 20-25° C.; suction filtration was conducted, and a filter cake was collected; 15 L of ethyl acetate was added to the filter cake for extraction and stirring for 20 min; and suction filtration was conducted, and an organic phase was collected. The filter cake was extracted and washed once again with 2 L ethyl acetate, and an organic phase was collected. The organic phases were combined; a product was concentrated under reduced pressure and then was slowly cooled; and a product was precipitated to obtain a crude product with a purity of 98.5% and an ee value of 98.6%. 2 times normal hexane by volume was added to the crude product for dissolution with heating; a product was crystallized with cooling; crystals were filtered and dried at a room temperature; 6.45 kg of white crystal product was collected and measured to have a purity of 99.70%, an ee value of 99.88% and a total yield of 86%.

Finally, it should be noted that the above embodiments are only used to illustrate the technical solution of the present invention and should not be considered as a limitation. Although the present invention has been described in detail with reference to the preferred embodiments, it should be understood by those skilled in the art that the modifications or equivalent substitutions could be made to the technical solution of the present invention without departing from the spirit and scope of the technical solution of the present invention and shall be all included within the scope of the claims of the present invention.

What is claimed is:

1-28. (canceled)

29. A composition, comprising:
a biological enzyme; and
a substrate, wherein:
the substrate is the β-carbonyl tetradecanoate which has the structural formula shown as I;
the biological enzyme is the ketoreductase having an amino acid sequence shown as SEQ ID NO: 1 or SEQ ID NO: 2;
a weight ratio of the biological enzyme to the substrate is 1:(1.1-150);
in the structural formula I, R is any one of methyl, ethyl, n-propyl or isopropyl; and

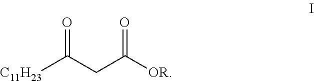

30. The composition in claim 29, wherein the biological enzyme is at least one of an enzyme powder, an enzyme solution and an immobilized enzyme.

31. The composition in claim 29, wherein the weight ratio of the biological enzyme to the substrate is one of 1:20, 1:30, 1:40, 1:50, 1:60, 1:70, 1:80, 1:90, 1:100, 1:110, 1:120, 1:130, 1:140 and 1:150.

32. A reaction system involving the composition in claim 29, comprising:
a substrate shown as I;
the biological enzyme;
glucose;
a glucose dehydrogenase;
NADP+; and
a buffer solution, wherein:
a sequence of the glucose dehydrogenase is shown as SEQ ID NO: 3;
in the structural formula I, R is any one of methyl, ethyl, n-propyl or isopropyl; and

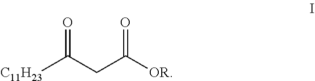

33. The reaction system in claim 32, wherein a pH value of the reaction system is 6.0-8.0.

34. The reaction system in claim 33, wherein the pH value of the reaction system is 7.0-7.5.

35. The reaction system in claim 32, wherein the buffer solution is a PBS buffer solution or a Tris-HCl buffer solution.

36. The reaction system in claim 32, wherein a concentration of the substrate is 20-150 g/L.

37. The reaction system in claim 32, wherein a concentration of NADP is 0.1-0.5 g/L.

38. The reaction system in claim 32, wherein a molar ratio of compound Ito the glucose is 1:(1.2-4).

39. The reaction system in claim 32, wherein a concentration of the buffer solution is 0.01-0.5 mol/L.

40. The reaction system in claim 32, wherein the reaction time of the reaction system does not exceed 15 hours before obtaining a reaction liquid.

41. A method for preparing an orlistat intermediate with the reaction system in claim 32, comprising the step of:
stirring the reaction system at 20-40° C. for reaction to obtain a reaction liquid of the orlistat intermediate, wherein:
an intermediate of the orlistat intermediate is (R)-β-hydroxytetradecanoate with a structural formula shown as II;
in the structural formula II, R is any one of the methyl, the ethyl, the n-propyl or the isopropyl; and

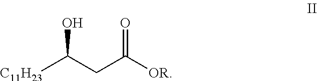

42. The method in claim 41, wherein a NaOH aqueous solution is used as a pH regulator.

43. The method in claim 42, a concentration of the NaOH aqueous solution is 2 M.

44. The method in claim 41, wherein the reaction time of the reaction system does not exceed 15 hours.

45. The method in claim 41, wherein a solvent is used in the reaction liquid to extract the orlistat intermediate.

46. The method in claim 45, wherein the solvent is anhydrous ethanol or ethyl acetate.

47. The method in claim 46, further comprising the steps of:
concentrating under reduced pressure the orlistat intermediate having been extracted;
cooling the concentrated orlistat intermediate and allowing it to crystalize; and
obtaining compound II which is a white crystalline solid, wherein:
in the structural formula II, R is any one of the methyl, the ethyl, the n-propyl or the isopropyl; and [chemical structure]


SEQUENCE LISTING (9 sequences)

SEQ ID NO: 1 - protein, 249 aa, Artificial Sequence
  Ketoreductase enzyme amino acid sequence SEQ ID NO 1
MGKLDNKVAVITGGNSGMGLATAQRFVSEGAYVFITGRRQAELDKAVDLIGKNVTGVQGDVSNLADLDRLYATVKEQKGRVDVLFANAGVGELAPLGSITEEQFDKVFNINVRGLLFTVQKALPLFQDGGSIILNASIASIKGMPAFSVYSASKAAVRSFARSWTVDLKGRKIRINTLSPGPIDTPILSGLASTEEELKQVKADLAAQVPLGRMGTSDEIANVALFLASDDSSYVTGIELFVDGGMAQI

SEQ ID NO: 2 - protein, 259 aa, Artificial Sequence
  Ketoreductase enzyme amino acid sequence SEQ ID NO 2
MARLAGKVALVTGGASVPGLGSATAIRFAQEGATVYLTDRDLAGAQAVAAQITAAGGRATALEHDVTSEADWDRVLAAIDAAEGRLDILVNNAGIAVLGPLEDVTAADFLRQNDVNLNSVFHGSKRALVMMRRPGDGGTARGGSIINISSVAGLIGVPGCGSYAASKGGVRLFSKVVALEGAADGVRCNSVHPGMIATNIQGVALEDNAANFDAVMALIPMVRMGEPEDIANMNLFLASDESRYITGAEFVVDGGMTAR

SEQ ID NO: 3 - protein, 263 aa, Artificial Sequence
  Glucose dehydrogenase
MYMYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRG

SEQ ID NO: 4 - protein, 248 aa, Artificial Sequence
  Ketoreductase enzyme amino acid sequence SEQ ID NO 4
MSSPAPTVYVISGASRGIGFAITSILAQHDNVLIFAGARDLKSAQLNELAQKSSGKVIPVKLESTSVEDAAALAKVVEEKAGKVDYVLAVAGISQSTDPIAQVSLDDVRRHFEVNTIGPLVLFQALLPLTTKSTAPHFIVVSTIAGSIASMPQVTFPVSAYAISKTAVNSAVGRIAIEHPDLDAFVCHPGFVSSDMVKQFAEKTGAPLSDFESFGMITPEESAASLVKLFDEAKKETHSGKFFNVDGT

SEQ ID NO: 5 - protein, 77 aa, Artificial Sequence
  Linker sequence
EFEEEEKKKQQEEEAERLRRIQEEMEKERKRREEDEERRRKEEEERRMKLEMEAKRKQEEEERKKREDDEKRKKKKL

SEQ ID NO: 6 - DNA, 1767 bp, Artificial Sequence
  Fusion Sequence coded Glucose dehydrogenase and Ketoreductase enzyme
atgtatatgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatattaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtgaattcgaagaagaggaaaaaaagaaacagcaggaagaagaagcggaacgtctgcgtcgtattcaggaggagatggagaaagaacgtaaacgccgcgaagaggacgaagaacgccgtcgtaaagaggaagaggagcgccgtatgaaactggaaatggaagcgaaacgcaaacaagaggaggaagagcgtaaaaagcgcgaggatgatgaaaaacgtaagaaaaaaaagcttatgtcttcgcctgctcccaccgtctacgtcatctctggcgcctctcgcggcatcggcttcgccatcacctccattctcgctcaacacgacaacgtattgatctttgccggcgcacgcgacctcaagtcggcgcaactgaacgagctcgctcagaagtctagcggcaaggtcatcccggtcaagctcgagtcgacgagtgtcgaggatgccgctgcgcttgccaaggtcgtcgaggagaaggctggaaaggtggactacgtcttggccgtcgccggcatctcccagtcgaccgaccccatcgcccaagtctccctcgacgacgtcaggcgtcacttcgaggtcaacaccatcggccctctcgtcctcttccaggcgctcctccccctcaccaccaagtcgaccgcgccgcacttcatcgtcgtctccaccatcgccggctcaatcgcctccatgcctcaagtcacgttccccgtgagcgcctacgctatctcgaagactgccgtcaactcggccgtaggacgaatcgcgatcgagcaccccgacctcgacgccttcgtctgccatccgggctttgtgagcagcgacatggtcaaacaatttgcggagaagacgggcgcaccgttgtcggactttgagtcgtttggcatgatcacccccgaagaatcggctgcgagtctcgttaagctgttcgacgaggccaagaaggagacgcactcgggcaagttctttaacgtcgacgggacctag

SEQ ID NO: 7 - DNA, 1779 bp, Artificial Sequence
  Fusion Sequence coding Ketoreductase enzyme and Glucose dehydrogenase
atgtcttcgcctgctcccaccgtctacgtcatctctggcgcctctcgcggcatcggcttcgccatcacctccattctcgctcaacacgacaacgtattgatctttgccggcgcacgcgacctcaagtcggcgcaactgaacgagctcgctcagaagtctagcggcaaggtcatcccggtcaagctcgagtcgacgagtgtcgaggatgccgctgcgcttgccaaggtcgtcgaggagaaggctggaaaggtggactacgtcttggccgtcgccggcatctcccagtcgaccgaccccatcgcccaagtctccctcgacgacgtcaggcgtcacttcgaggtcaacaccatcggccctctcgtcctcttccaggcgctcctccccctcaccaccaagtcgaccgcgccgcacttcatcgtcgtctccaccatcgccggctcaatcgcctccatgcctcaagtcacgttccccgtgagcgcctacgctatctcgaagactgccgtcaactcggccgtaggacgaatcgcgatcgagcaccccgacctcgacgccttcgtctgccatccgggctttgtgagcagcgacatggtcaaacaatttgcggagaagacgggcgcaccgttgtcggactttgagtcgtttggcatgatcacccccgaagaatcggctgcgagtctcgttaagctgttcgacgaggccaagaaggagacgcactcgggcaagttctttaacgtcgacgggaccgaattcgaagaagaggaaaaaaagaaacagcaggaagaagaagcggaacgtctgcgtcgtattcaggaggagatggagaaagaacgtaaacgccgcgaagaggacgaagaacgccgtcgtaaagaggaagaggagcgccgtatgaaactggaaatggaagcgaaacgcaaacaagaggaggaagagcgtaaaaagcgcgaggatgatgaaaaacgtaagaaaaaaaagcttatgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatattaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtcaccaccaccaccaccactag

SEQ ID NO: 8 - protein, 588 aa, Artificial Sequence
  Fusion enzyme of Glucose dehydrogenase and Ketoreductase enzyme
MYMYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRGEFEEEEKKKQQEEEAERLRRIQEEMEKERKRREEDEERRRKEEEERRMKLEMEAKRKQEEEERKKREDDEKRKKKKLMSSPAPTVYVISGASRGIGFAITSILAQHDNVLIFAGARDLKSAQLNELAQKSSGKVIPVKLESTSVEDAAALAKVVEEKAGKVDYVLAVAGISQSTDPIAQVSLDDVRRHFEVNTIGPLVLFQALLPLTTKSTAPHFIVVSTIAGSIASMPQVTFPVSAYAISKTAVNSAVGRIAIEHPDLDAFVCHPGFVSSDMVKQFAEKTGAPLSDFESFGMITPEESAASLVKLFDEAKKETHSGKFFNVDGT

SEQ ID NO: 9 - protein, 592 aa, Artificial Sequence
  Fusion enzyme of Ketoreductase enzyme and Glucose dehydrogenase
MSSPAPTVYVISGASRGIGFAITSILAQHDNVLIFAGARDLKSAQLNELAQKSSGKVIPVKLESTSVEDAAALAKVVEEKAGKVDYVLAVAGISQSTDPIAQVSLDDVRRHFEVNTIGPLVLFQALLPLTTKSTAPHFIVVSTIAGSIASMPQVTFPVSAYAISKTAVNSAVGRIAIEHPDLDAFVCHPGFVSSDMVKQFAEKTGAPLSDFESFGMITPEESAASLVKLFDEAKKETHSGKFFNVDGTEFEEEEKKKQQEEEAERLRRIQEEMEKERKRREEDEERRRKEEEERRMKLEMEAKRKQEEEERKKREDDEKRKKKKLMYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRGHHHHHH